(19) United States
(12) Patent Application Publication
(10) Pub. No.: US 2018/0216142 A1
(43) Pub. Date: Aug. 2, 2018

(54) PROCESS FOR ENZYMATIC PRODUCTION OF OXIDATION AND REDUCTION PRODUCTS OF MIXED SUGARS

(71) Applicant: ANNIKKI GMBH, Raaba-Gramach (AT)

(72) Inventors: Ortwin Ertl, Vasoldsberg (AT); Bernd Mayer, Graz (AT); Alexander Dybov, Seiersberg-Pirka (AT)

(21) Appl. No.: 15/747,262

(22) PCT Filed: Jul. 20, 2016
(86) PCT No.: PCT/EP2016/067287

§ 371 Date: Jan. 24, 2018

(30) Foreign Application Priority Data
Jul. 24, 2015 (EP) 15178205.9

(51) Int. Cl. C12P 7/58; C12P 7/18; D21C 3/20; C07H 3/02; C12N 9/04

(52) U.S. Cl. CPC C12P 7/58; C12P 7/18; D21C 3/20; C07H 3/02; C12N 9/0006; C12Y 101/01179; C12Y 101/03004; C12Y 101/01116; C12Y 101/01117; C12Y 101/01175; C12P 2201/00

(57) ABSTRACT

The present invention provides a process for obtaining n+a oxidation and reduction products from a mixture of n sugars selected from the group consisting of C5 and C6 sugars,
wherein n is at least 2 and a is at least 1,
wherein at least two of the sugars in the mixture are present at a non-equimolar ratio to each other,
wherein, in a first stage, at least one of the sugars which are present at a non-equimolar ratio to each other is oxidized enzymatically and, at the same time, at least one of the other sugars present at a non-equimolar ratio to each other is reduced enzymatically, and
wherein, in the first stage, a portion of at least one of the sugars present at a non-equimolar ratio to each other is not converted,
and which is characterized in that, in at least a second stage, at least a portion of the sugar not converted in the first stage is oxidized enzymatically by half and, respectively, is reduced enzymatically by the remaining half.

The present invention relates to a process for obtaining n+a oxidation and reduction products from a mixture of n sugars selected from the group consisting of C5 and C6 sugars.

Many methods for the fractionation of sugar mixtures have been published in the literature. Many of them are based on chromatography.

In U.S. 2004/0173533 A1, a method for the chromatographic separation of a mixture consisting of sugars, preferably of xylose and glucose, has been described. The separation yields separate streams: one of them is enriched with xylose, the other one is enriched with glucose. The sugar mixture is preferably produced by hydrolysis of biomass.

A separation of sugars, sugar alcohols, carbohydrates and mixtures thereof is described in EP 1 490 521 B1, wherein, in at least one step, a weakly basic anion exchange resin (crosslinked polyacrylic acid polymer or epichlorohydrin-triethylenetetramine resin) is used in the chromatographic separation.

From CA 2 359 337, a process for the separation of sugars is known, wherein the separation of xylose, mannose, galactose, arabinose, glucose, xylitol, arabitol, sorbitol, galactitol or mannitol (or other monosaccharides) from other sugars or sugar alcohols is effected by chromatography via an ion exchanger which has been produced from an anion exchanger having a relatively low content of hydroxyl.

A major drawback of chromatographic sugar fractionations is the incomplete separation of components. As a consequence, low yields and an increased share of other sugars in the main sugar fraction are to be mentioned.

Another approach to the fractionation of sugar is an (optionally) selective sugar conversion into components which can be separated easily from each other as a result of different physical or chemical properties (polarity, solubility etc.).

An example of such a fractionation is described in U.S. Pat. No. 7,498,430, wherein the fractionation of xylose and arabinose is effected from a mixture of sugars. The fractionation happens by converting the sugars into a mixture of xylose monoacetal and arabinose diacetal. Subsequently, xylose monoacetal is separated from arabinose diacetal by liquid-liquid extraction.

In WO 2011/133536 A1, a process is described wherein C5 and/or C6 aldose sugar in sugar hydrolysate is contacted with a catalyst in order to convert the sugars into ketose isomers. Furthermore, the isomerized C5 and/or C6 ketoses have been contacted with a complexing agent (CA), whereby the ketoses bind to the CA and a ketose-CA conjugate emerges. The ketose-CA conjugate could be separated selectively from the sugar mixture.

In the present invention, a portion of the sugars in the sugar mixture is converted selectively into sugar acids as a first step toward sugar fractionation. Several methods for the production of sugar acids have been described in the literature.

A process for the production of gluconic acid is known from U.S. Pat. No. 2,651,592. A glucose solution is added to an enzymatic system which exhibits catalase and glucose oxidase activity, wherein hydrogen peroxide is added at a stoichiometric amount in order to oxidize the entire glucose.

In U.S. Pat. No. 3,619,396, a method is described wherein gluconic acid is produced enzymatically from a glucose-containing material by means of glucose oxidase. An improvement over known methods consists in that the reaction medium is subjected to electrodialysis in order to separate gluconic acid from the medium and to recover glucose oxidase.

In U.S. Pat. No. 3,935,071, glucose is converted into gluconic acid, wherein glucose is oxidized with oxygen in an aqueous solution. A glucose solution is passed through a catalyst which contains glucose oxidase and catalase and is bound firmly to an appropriate carrier. In an example, a selective oxidation of glucose in the presence of fructose is described, wherein the nascent gluconic acid is subsequently separated by means of an ion exchanger.

A method of producing gluconic acid and its salts is known from CA 2 194 859, wherein glucose having a concentration of 15% or more is converted into gluconic acid at a temperature of 10° C. or more in the presence of glucose oxidase and catalase. The conversion is carried out such that an excess of catalase activity relative to oxidase activity is used.

In U.S. Pat. No. 7,923,226 B2, a process for the production of 1,2,4-butanetriol is described, wherein xylose is also oxidized to xylonolactone/xylonic acid. However, this patent fails to disclose a recycling system for the redox cofactor reduced in the reaction.

Also for the production of sugar acids, fermentative methods are already employed (e.g., Buchert et al., 1988; Toivari et al., 2012b).

In U.S. Pat. No. 2,351,500, a method is described wherein glucose is converted into gluconic acid by fermentation. Boric acid is added in an amount of from 0.25 to 1.5 relative to the nascent gluconic acid in order to prevent the precipitation of gluconic acid salts.

In the literature, there are also several examples of fermentative methods in which both xylonic acid and xylitol are formed from xylose. In Nygard et al. (2011), Kluyveromyces lactis strains are used in order to convert xylose into xylonic acid and xylitol. However, a large amount of biomass is produced in the process (approx. 50% relative to the sum of the products xylonic acid and xylitol). In further prior art methods, S. cerevisiae strains (Toivari et al., 2012b) or modified E. coli (Cao et al., 2013) are used for producing xylonic acid and xylitol from xylose. Also in that case, a very large amount of biomass is produced, whereby substances are also consumed which are added to the medium in addition to xylose, such as, for example, glucose.

In the described methods and in further fermentative methods for the production of xylonate according to the prior art, the following difficulties become apparent: losses as a result of the production of biomass, acetate or other sugar acids, a relatively low substrate concentration and/or relatively long reaction times.

Several enzymatic methods of producing the sugar alcohol xylitol using isolated enzymes are known (e.g., Zhang et al. (2011)). In those methods, the employed redox cofactor is regenerated by a further enzymatic redox reaction. However, for this purpose, an additional substrate must be added in each case at an at least stoichiometric amount. Nidetzky et al. (1996) have described a method wherein xylitol is produced from xylose and wherein the reduction equivalents are provided by the simultaneous oxidation of either glucose or xylose.

A further well-known possibility of producing the sugar alcohol xylitol is fermentation. In Ko et al. (2006), for example, a fermentation process for obtaining xylitol from xylose is described. Thereby, a genetically engineered strain of Candida tropicalis is used. The regeneration of the cofactor of xylose reductase is not specified any further and is taken over by the overall metabolism of the cells. A disadvantage which becomes apparent is that glucose is additionally added to the cultures. A large part of the sugars used is converted into biomass and does not serve for the formation of the product. Most notably, the stoichiometrically possible amount of xylitol relative to the employed xylose is not obtained. Enzymes are described in the literature which enable the conversion of L-arabonate into alpha-ketoglutarate via L-2-keto-3-deoxyarabonate and alpha-ketoglutarate semialdehyde (Watanabe et al., 2006). Furthermore, enzymes are described which enable the conversion of D-xylonic acid into alpha-ketoglutarate via D-2-keto-3-deoxyxylonate and alpha-ketoglutarate semialdehyde (Stephens et al., 2006; Johnsen et al., 2009).

In WO 2014/076012 A1, a method is described among other things in which, from a mixture of arabinose and xylose (at a molar ratio of about 10 to 90), the arabinose is oxidized largely enzymatically to arabinolactone or arabonic acid, respectively, and the xylose is reduced enzymatically to xylitol at an essentially equimolar ratio.

The described molar ratio between arabinose and xylose is typical of a mixture of sugars which may be obtained by pulping a lignocellulose-containing biomass and subsequently enzymatically degrading the hemicellulose-containing material obtained by the pulping.

Further prior art is known from WO 2010/106230 A1.

It is the object of the present invention to fractionate the very complex sugar mixtures, which often emerge from biomass, at a high purity and a high yield, irrespective of the number and structure of the sugars. Furthermore, it is an object of the present invention to provide a possibility for the further conversion of fractionated sugars/sugar acids, in particular xylose, xylonic acid, arabinose, arabonic acid, into further products, in particular xylitol and α-ketoglutarate.

The object of the present invention is achieved by a process according to claim 1.

Preferred embodiments of the present invention are indicated in the subclaims.

First of all, the present invention provides a process for obtaining n+a oxidation and reduction products from a mixture of n sugars selected from the group consisting of C5 and C6 sugars,
wherein n is at least 2 and a is at least 1,
wherein at least two of the sugars in the mixture are present at a non-equimolar ratio to each other,
wherein, in a first stage, at least one of the sugars which are present at a non-equimolar ratio to each other is oxidized enzymatically and, at the same time, at least one of the other sugars present at a non-equimolar ratio to each other is reduced enzymatically, and
wherein, in the first stage, a portion of at least one of the sugars present at a non-equimolar ratio to each other is not converted,
and which is characterized in that, in at least a second stage, at least a portion of the sugar not converted in the first stage is oxidized enzymatically by half and, respectively, is reduced enzymatically by the remaining half.

Preferably, in the second stage, essentially the entire amount of the sugar not converted in the first stage is oxidized enzymatically by half and, respectively, is reduced enzymatically by the remaining half.

Furthermore, the two sugars which have been oxidized and reduced, respectively, in the first stage are preferably converted at an essentially equimolar amount.

The process according to the invention is based on the coupling of enzymatic oxidation and reduction reactions on sugars in such a way that sugar oxidation products (such as, e.g., sugar acids) and sugar reduction products (such as, e.g., sugar alcohols), which are separable from each other, are obtained from a sugar mixture.

In many sugar mixtures obtained in particular from biomass, the sugars contained therein are not present at an equimolar ratio. If—as described, for example, in WO 2014/076012 A1—an enzymatic oxidation of one sugar and an enzymatic reduction of the other sugar occur at an essentially equimolar ratio in a first stage, a portion of the sugar which is present in the original mixture in a larger amount will remain in the mixture in an unreacted form.

According to the invention, this unreacted portion of said sugar will now also be subjected to enzymatic oxidation and enzymatic reduction. As a result, an oxidation product (such as, e.g., a sugar acid) as well as a reduction product (such as, e.g., a sugar alcohol) of said sugar, which can be separated from each other, will again emerge at an essentially equimolar ratio.

If the entire portion of said sugar which has not been converted in the first stage is oxidized and reduced, a mixture results which consists exclusively of those oxidation and reduction products, and a complete separation of the products is thus possible.

Thus, for example, in a mixture of two sugars (n=2), three (a=1) products will emerge according to said process, namely the oxidation product of the sugar oxidized in the first stage as well as the oxidation and reduction product of the other sugar.

In particular if a mixture of more than two sugars is present, the first stage may comprise several substeps:

For example, in a mixture of three sugars A, B and C, wherein sugar C is present at a molar excess, sugar A can be oxidized or reduced completely in a first substep of the first stage and, correspondingly, sugar C can be reduced or oxidized partly at an equimolar ratio. In a second substep, for example, sugar B can be oxidized or reduced completely and, again, a portion of sugar C can be reduced or oxidized correspondingly. Then, in the second stage, the entire unreacted portion of sugar C would preferably be reduced and oxidized, in each case by half.

The substeps of the first stage can proceed simultaneously or also consecutively.

According to the invention, sugar acids and sugar acid lactones, respectively, are preferably obtained as oxidation products, and sugar alcohols are obtained as reduction products.

In a process according to the present invention, preferably a mixture of substances containing xylose and at least one further sugar preferably selected from the group consisting of C5 sugars, such as, e.g., arabinose, lyxose, ribose, and C6 sugars, such as, e.g., allose, altrose, glucose, mannose, idose, galactose and talose, is converted.

In a particularly preferred embodiment, the mixture of sugars contains xylose and arabinose, with xylose being present in excess.

Such mixtures accumulate in particular during the decomposition of a hemicellulose-containing material which has been obtained by pulping a lignocellulosic material, in particular if the lignocellulosic material is a material selected from the group consisting of straw, in particular wheat straw, bagasse, energy grasses, in particular elephant grass, switch grass, and/or husks, in particular lemmas.

Typically, the molar ratio of xylose and arabinose in mixtures obtained in this way may amount to approx. 9:1.

In an embodiment in which a sugar mixture with an excess amount of xylose and arabinose is present, arabinose is preferably oxidized to arabonic acid or to arabonic acid lactone, respectively, and a portion of the xylose is reduced to xylitol in the first stage, and, in the second stage, the unreacted xylose is oxidized completely or partly to xylonic acid or to xylonolactone by half and, respectively, the remaining half is reduced to xylitol.

In said aspect of the present invention, no additional sugars must be added to the mixture in order to produce xylitol, for example, since the required oxidation equivalents are provided through the simultaneous oxidative production of the arabonic acid.

Starting from an exemplary ratio of xylose to arabinose of 9:1 in the mixture, the following sequence, for example, ensues thereby:

If, in such a mixture, essentially the entire amount of arabinose is oxidized to arabonic acid and an essentially equimolar amount of xylose is reduced to xylitol in a first stage, a mixture of 1 part of arabonic acid, 1 part of xylitol and 8 parts of unreacted xylose is obtained.

If, in the second stage, preferably the entire unreacted xylose is now reduced enzymatically to xylonic acid by half and to xylitol by half, a mixture of 1 part of arabonic acid, 4 parts of xylonic acid and 5 parts of xylitol will ensue (provided that the arabonic acid has not already been separated beforehand).

Thus, from the orginal sugar mixture, a mixture of easily separable oxidation and reduction products arises in an elegant fashion, which products either already constitute valuable substances by themselves (such as, for example, xylitol) or can be processed further into valuable products.

Thus, in this embodiment of the present invention, arabonic acid which has formed and/or xylonic acid which has formed is/are preferably processed further into α-ketoglutaric acid.

Also this further processing may preferably occur enzymatically:

For the enzymatic conversion of the sugar acids arabonic acid and xylonic acid into alpha-ketoglutarate, in case of arabonic acid, said acid can be converted first into L-2-keto-3-deoxyarabonate by means of an arabonic acid dehydratase, then into alpha-ketoglutaric acid semialdehyde (alpha-KGSA) by means of an L-2-keto-3-deoxyarabonate dehydratase and further into alpha-ketoglutarate by means of an alpha-KGSA dehydrogenase. In case of xylonic acid, said acid can be converted first into D-2-keto-3-deoxyxylonate by means of a xylonic acid dehydratase, then into alpha-ketoglutaric acid semialdehyde (alpha-KGSA) by means of a D-2-keto-3-deoxyxylonate dehydratase and further into alpha-ketoglutarate by means of an alpha-KGSA dehydrogenase.

Suitable representatives of the enzyme classes L-arabonic acid dehydratase and L-2-keto-3-deoxyarabonate dehydratase are obtainable, for example, from Azospirillum brasiliense. Suitable representatives of the enzyme classes xylonic acid dehydratase and D-2-keto-3-deoxyxylonate dehydratase are obtainable, for example, from Caulobacter crescentus. Suitable alpha-ketoglutaric acid semialdehyde dehydrogenases are obtainable, for example, from Azospirillum brasiliense or from Caulobacter crescentus.

A further preferred embodiment of the process according to the invention is characterized in that the mixture containing xylose and arabinose additionally contains glucose.

In particular in said embodiment of the process, the glucose contained in the mixture is oxidized to gluconic acid.

In said embodiment of the process, arabinose is preferably oxidized enzymatically to arabonic acid, preferably after the separation of the gluconic acid.

Starting from an exemplary mixture of xylose/arabinose/glucose at a ratio of 9:1:0.4 (a mixture in that range can be obtained, for example, during the pulping of straw and the subsequent enzymatic decomposition), the following sequence, for example, ensues thereby:

If, in such a mixture, essentially the entire amount of glucose is oxidized to gluconic acid and an essentially equimolar amount of xylose is reduced to xylitol in a first stage, a mixture of 1 part of arabinose, 0.4 parts of gluconic acid, 0.4 parts of xylitol and 8.6 parts of unreacted xylose is obtained.

If, in a further reaction of the first stage, essentially the entire amount of arabinose is oxidized to arabonic acid and again an essentially equimolar amount of xylose is reduced to xylitol, a mixture of 1 part of arabonic acid, 0.4 parts of gluconic acid, 1.4 parts of xylitol and 7.6 parts of unreacted xylose is obtained.

If, in the second stage, preferably the entire unreacted xylose is now oxidized enzymatically to xylonic acid by half and, respectively, reduced to xylitol by half, a mixture of 1 part of arabonic acid, 0.4 parts of gluconic acid, 3.8 parts of xylonic acid and 5.2 parts of xylitol will ensue (provided that the arabonic acid and/or the gluconic acid has/have not already been separated beforehand).

Further processing of the resulting arabonic acid and/or xylonic acid into alpha-ketoglutarate is feasible as described above.

In a further preferred embodiment, the sugar mixture contains glucose in excess relative to the other existing sugar(s), and sorbitol is obtained at least partly from the glucose.

The starting point should be an exemplary mixture of glucose/mannose/galactose/xylose/arabinose at a ratio of 7:1.4:0.7:0.7:0.4. A mixture in that range can be obtained, for example, during the pulping of wood and the subsequent enzymatic degradation (Berrocal et al. 2004). A sugar mixture having a high content of glucose can also be obtained during the complete hydrolysis of sugar polymers from other lignocellulose-containing biomasses such as, for example, straw, corn straw, rice straw, bagasse, energy grasses. For example, the following sequence ensues thereby:

If, in such a mixture, essentially the entire amount of mannose is oxidized to mannonic acid and an essentially equimolar amount of glucose is reduced to sorbitol in a first stage, a mixture of 1.4 parts of mannonic acid, 0.7 parts of galactose, 0.7 parts of xylose, 0.4 parts of arabinose, 1.4 parts of sorbitol as well as 5.6 parts of unreacted glucose is obtained.

If, in a further reaction of the first stage, essentially the entire amount of galactose is oxidized to galactonic acid and again an essentially equimolar amount of glucose is reduced to sorbitol, a mixture of 1.4 parts of mannonic acid, 0.7 parts of galactonic acid, 0.7 parts of xylose, 0.4 parts of arabinose, 2.1 parts of sorbitol as well as 4.9 parts of unreacted glucose is obtained.

If, in a further reaction of the first stage, essentially the entire amount of xylose is oxidized to xylonic acid and again an essentially equimolar amount of glucose is reduced to sorbitol, a mixture of 1.4 parts of mannonic acid, 0.7 parts of galactonic acid, 0.7 parts of xylonic acid, 0.4 parts of arabinose, 2.8 parts of sorbitol as well as 4.2 parts of unreacted glucose is obtained.

If, in a further reaction of the first stage, essentially the entire amount of arabinose is oxidized to arabonic acid and again an essentially equimolar amount of glucose is reduced to sorbitol, a mixture of 1.4 parts of mannonic acid, 0.7 parts of galactonic acid, 0.7 parts of xylonic acid, 0.4 parts of arabonic acid, 3.2 parts of sorbitol as well as 3.8 parts of unreacted glucose is obtained.

The steps of the first stage can proceed consecutively or (partly) simultaneously. In addition, a separation of sugar acids may occur after individual steps.

If, in the second stage, preferably the entire unreacted glucose is now oxidized enzymatically to gluconic acid by half and, respectively, reduced to sorbitol by half, a mixture of 1.4 parts of mannonic acid, 0.7 parts of galactonic acid, 0.7 parts of xylonic acid, 0.4 parts of arabonic acid, 1.9 parts of gluconic acid and 5.1 parts of sorbitol will ensue (provided that the sugar acids have not already been separated beforehand).

A separation of sugar acids may (also) occur after the second stage. Further processing of the resulting arabonic acid and/or xylonic acid into alpha-ketoglutarate is feasible as described above.

In a possible optional further stage, the obtained D-sorbitol can be oxidized enzymatically or non-enzymatically, preferably enzymatically, to D-fructose, for example, with a D-sorbitol dehydrogenase or with an enzyme which has D-sorbitol dehydrogenase activity. Redox cofactors NAD(P), which possibly have been reduced by the enzyme, can be regenerated by at least one further redox enzyme. As a result, the redox cofactors can be used at a substoichiometric amount.

In said specific embodiment, the process according to the present invention may be used, for example, for obtaining a very pure D-fructose from a biomass hydrolysate with a glucose content. Easier separation of the other sugars from the mixture is enabled by the conversion into sugar acids. Furthermore, the oxidation to sugar acids provides redox equivalents for the reduction of glucose to sorbitol. In the above-described exemplary mixture, a relatively high proportion of the glucose in the mixture can be converted into sorbitol without adding external substances to the redox cofactor recycling. The sorbitol thereby obtained is available for the preparation of the valuable product fructose.

The enzymatic oxidation of sugars as performed in the process according to the invention can be effected by various enzyme classes. For example, oxidases (using oxygen) or dehydrogenases (using the oxidized redox cofactors NAD(P)⁺) are suitable for this purpose. Preferably, enzymes are used which are dependent on redox cofactors. Particularly preferably, enzymes solely dependent on redox cofactors are used. Thus, an oxidation and a reduction, respectively, of the affected sugars, in each case proceeding at an equimolar ratio, can be achieved in an elegant fashion. Accordingly, a further preferred embodiment of the process according to the invention is characterized in that, at least in one of the two stages, preferably at least in the second stage, particularly preferably both in the first and in the second stage, at least one redox cofactor and at least one enzyme dependent on said redox cofactor are present in the reaction mixture.

In a process according to the present invention, preferably arabinose is oxidized to arabonic acid, particularly in the first stage. For example, an L-arabinose dehydrogenase may be used for the oxidation of L-arabinose. Suitable L-arabinose dehydrogenases are obtainable, for example, from Azospirillum brasiliense or from Burkholderia vietnamiensis.

In a process according to the present invention, preferably xylose, in particular a portion of the xylose remaining in the solution after the first stage, is oxidized to xylonic acid. The oxidation of D-xylose can be effected, for example, by means of a D-xylose dehydrogenase. Suitable xylose dehydrogenases are obtainable, for example, from Caulobacter crescentus. Alternatively, enzymes having a broader substrate spectrum and annotated as arabinose dehydrogenases may also be used.

In a process according to the present invention, preferably glucose is oxidized to gluconic acid, particularly in the first stage. For example, a D-glucose-1-dehydrogenase may be used for the oxidation of glucose. A suitable D-glucose-1-dehydrogenase is obtainable, for example, from Bacillus subtilis.

Furthermore, a glucose oxidase may be used for the oxidation of glucose. A suitable D-glucose oxidase is obtainable, for example, from Aspergillus niger.

In a process according to the present invention, preferably xylose, in particular a portion of the xylose remaining in the solution after the first stage, is reduced to xylitol.

For example, the reduction of D-xylose can be catalyzed by a D-xylose reductase. Suitable xylose reductases are obtainable, for example, from Candida tropicalis, Candida parapsilosis or Saccharomyces cerevisiae.

In a process according to the present invention, the cofactors NADH, NADPH, NAD⁺ and/or NADP⁺ may be used in specific embodiments. Thereby, NAD⁺ denotes the oxidized form and NADH denotes the reduced form of nicotinamide adenine dinucleotide, while NADP⁺ denotes the oxidized form and NADPH denotes the reduced form of nicotinamide adenine dinucleotide phosphate. The cofactors either may be added to the reaction seperately, or they are part of other components of the reaction, for example, of the enzymes used, or a combination of those two sources is used. If redox cofactors are used, they are present in substoichiometric amounts relative to the substrates in a process according to the present invention. The redox cofactors oxidized or reduced in reduction or oxidation reactions can be returned to their original redox state via suitable enzymatic reactions (redox cofactor recycling) and thus can pass through several reaction cycles.

Enzymatic cofactor regeneration systems are selected in particular from the group consisting of alcohol dehydrogenases, sugar dehydrogenases, NAD(P)H oxidases, hydrogenases or lactate dehydrogenases, whereby cosubstrates, in particular ketones, aldehydes, sugars, pyruvic acid and its salts and/or oxygen, is/are consumed and, respectively, hydrogen is produced.

In the production of xylonic acid (lactone) from xylose, redox cofactor recycling can be effected by a further redox enzyme, for example, by an alcohol dehydrogenase, a NAD(P)H oxidase or a sugar reductase such as, e.g., a xylose reductase. Preferably, a sugar reductase is used.

In the production of xylitol, redox cofactor recycling can likewise be effected by a further redox enzyme, for example, by an alcohol dehydrogenase or a sugar dehydrogenase such as, e.g., a glucose dehydrogenase or an arabinose dehydrogenase. Preferably, a sugar dehydrogenase is used.

Suitable NADH oxidases are obtainable, for example, from Clostridium aminovalericum or Streptococcus mutans.

Suitable alcohol dehydrogenases are obtainable, for example, from Lactobacillus kefir or Thermoanaerobium brockii.

In a specific embodiment of the process according to the present invention, glucose is converted into gluconic acid (lactone) with a glucose dehydrogenase, with redox cofactor recycling being effected by a xylose reductase. In a further specific embodiment of the process according to the present invention, the nascent gluconic acid is separated from the mixture of substances.

In a further specific embodiment of the process according to the present invention, arabinose is converted into arabonic acid (lactone) with an arabinose dehydrogenase, with redox cofactor recycling being effected by a xylose reductase. In a further specific embodiment of the process according to the present invention, the resulting arabonic acid is separated from the mixture of substances. In another preferred embodiment of the process according to the present invention, arabonic acid (lactone) is not separated from the mixture of substances, whereupon the remaining xylose is oxidized to xylonic acid (lactone).

In an aspect of the present invention, the xylose remaining after the first stage is reduced to xylitol both in the presence of sugar acids which are forming and, optionally, also after the separation of sugar acids, namely with an enzyme, preferably with a xylose reductase, more preferably with a NAD(P)-dependent xylose reductase. In another aspect, the remaining xylose is reduced to xylitol with a NAD(P)-dependent xylose reductase, with redox cofactor recycling being effected by a xylose dehydrogenase so that the remaining xylose is simultaneously oxidized to xylonic acid.

Thus, in the process according to the invention, both in the first and in the second stage, at least one redox cofactor and at least one enzyme dependent on said redox cofactor are preferably present in the reaction mixture. Usually, two enzymes each are present in the two stages, for example, a reductase on the one hand and a dehydrogenase on the other hand. However, catalysis of both the reduction and the oxidation by a single enzyme is also conceivable.

As has already been mentioned above, particularly preferably all enzymes used are dependent on a redox cofactor which is also present in the mixture.

Either the redox cofactor is already contained in the enzyme preparations at a sufficient amount, or redox cofactor is additionally added to the reaction.

Furthermore, the redox cofactor used in the first and/or second stage is preferably regenerated in the process according to the invention by reduction and oxidation reactions which, in each case, proceed in parallel.

The enzymes used in the process can be obtained by recombinant expression. In this connection, various systems are known to a person skilled in the art, for example, E. coli, Saccharomyces cerevisiae or Pichia pastoris. Preferably, E. coli is used; for this purpose, a person skilled in the art is familiar with common protocols. The enzymes can be used in intact cells, in permeabilized cells or in the form of cell lysates. In the case of cell lysates, either the enzymes may be used directly, or a further purification may occur, for example, by chromatographic methods for protein purification, which may be found in the literature and/or are known to a person skilled in the art. If cell lysates are used, either no further purification or only a simple purification step (e.g., centrifugation or filtration) is preferably performed.

As has already become apparent from the previous explanations, the first and the second stages in the process according to the invention can be performed in a one-pot reaction.

Thus, a complete separation of the sugar mixture can possibly be achieved in only one reaction vessel.

In a further preferred embodiment of the process according to the invention, the two stages can proceed at least partly simultaneously. Based on the enzymatic reactions as described, a simultaneous reaction control of both stages is possible.

A further embodiment of the process according to the invention comprises the removal of accumulating sugar acids from the mixture. In doing so, the removal of accumulating sugar acids (e.g., arabonic acid, gluconic acid or xylonic acid) may occur between the first and the second stages of the process according to the invention, or else only after the second stage.

As mentioned above, in the case of a mixture of more than two sugars, it is possible to oxidize several sugars in the first stage of the process according to the invention, namely in substeps of the first stage, which proceed in parallel or consecutively. If the substeps are performed consecutively, it is possible in a specific embodiment of the process according to the invention to separate the respective sugar acid after each substep. Optionally, the sugar acids may also be separated jointly after the first stage, or, alteratively, they may be left in the solution.

Several methods for the separation of organic acids are known to a person skilled in the art from the prior art. Said methods include, but are not limited to, ion-exchange chromatography, electrodialysis, crystallization/precipitation and extraction.

As has already been mentioned above, the mixture containing the sugars can be obtained from a hemicellulose-containing material.

Preferably, the hemicellulose-containing material has been obtained by pulping a lignocellulosic material. In this connection, various chemical, physical, mechanical and/or enzymatic methods are known to a person skilled in the art. Pulping methods for a lignocellulose-containing material can also be found in Brodeur et al. (2011). Furthermore, a method for the pulping or delignification, respectively, of a lignocellulose-containing material can be found, for example, in WO 2010/124312 A2 (Ertl et al., 2010).

In a process according to the present invention, a “lignocellulose-containing material” includes in particular a lignocellulose-containing biomass, e.g., annual or perennial plants or parts of annual or perennial plants such as, for example, wood such as, e.g., softwood or hardwood, or (dry) grasses, or parts of grasses, preferably grasses, straw such as, e.g., wheat straw, rye straw or corn straw, energy grasses such as, e.g., Panicum virgatum, miscanthus/Chinese silvergrass, abaca, sisal, bagasse, or atypical lignocellulose substrates such as corn cobs, husks, e.g., lemmas, such as wheat husks, rice husks, particularly preferably straw, in particular wheat straw, bagasse, energy grasses, in particular elephant grass, switch grass, and/or husks, in particular lemmas.

Preferably, the lignocellulosic material can be obtained by pulping with an alcohol, in particular with a C₁₋₄ alcohol, water and an alkali. An appropriate method is known, for example, from WO 2010/124312 A2 (Ertl et al., 2010).

In a process according to the present invention, if acids are indicated, the corresponding salts of those acids are included and vice versa. Furthermore, specifically if enzymatically produced sugar acids are indicated, the corresponding sugar acid lactones are also included and vice versa. In the enzymatic oxidation of sugars to sugar acids/sugar acid lactones, the ratio of those two products depends strongly on the sugar which is used and on the reaction conditions such as, for example, the reaction time or especially the pH-value.

### EXAMPLES

### Example 1: Xylanase Treatment for Obtaining a Sugar Mixture from Biomass

Delignified pulp produced from straw was used. A description of the preparation of the pulp can be found in WO 2010/124312 A2 (Example 1). 10 g (dry weight) of the pulp was resuspended with distilled water to a consistency of 10%, and a pH-value of 4.9 was adjusted with H₂SO₄. 1000 μl of Xylanase Ecopulp TX800A (Ecopulp Finland Oy) was added, and incubation took place at 50° C. for 16 h. A 1.5% sugar solution (w/v) is obtained which contains mainly glucose, xylose and arabinose at a ratio of about 2:10:1.

The following examples 2 to 5 serve for illustrating the possibilities for a selective enzymatic oxidation or reduction, respectively, of sugars from a sugar mixture.

### Example 2: Glucose Oxidation with a Glucose Dehydrogenase (NADH-oxidase for Cofactor Recycling)

18.5 mg NaHCO₃ was added to a sugar mixture (approx. 500 μl) containing glucose, xylose and arabinose (sugar concentration: approx. 1%). Thereupon, 30 μl glucose dehydrogenase (activity of approx. 300 U/ml), 10 μl NADH-oxidase (activity of approx. 1140 U/ml) and 2.5 μl NADH (concentration: 100 mM) were added. The mixture was incubated at 25° C. for approx. 17 hours. 86% of the glucose was reacted to gluconic acid. The obtained solution was passed through a strong ion exchanger (Amberlyst A-26(OH), Alfa Aesar). Thereby, the resulting gluconic acid was separated completely from the mixture.

### Example 3: Arabinose Oxidation with an Arabinose Dehydrogenase (NADH-oxidase for Cofactor Recycling)

6.2 mg NaHCO₃ was added to a sugar mixture (approx. 500 μl) containing glucose, xylose and arabinose (sugar concentration: approx. 1%). Thereupon, 30 μl arabinose dehydrogenase (activity of approx. 300 U/ml), 20 μl NADH-oxidase (activity of approx. 1140 U/ml) and 2.5 μl NADH (concentration: 100 mM) were added. The mixture was incubated at 25° C. for approx. 17 hours. 100% of the arabinose was reacted to arabonic acid. The obtained solution was passed through a strong ion exchanger (Amberlyst A-26(OH), Alfa Aesar). Thereby, the resulting arabonic acid was separated completely from the sugar mixture.

### Example 4: Arabinose Oxidation with an Arabinose Dehydrogenase (Xylose Reductase for Cofactor Recycling)

16.9 mg NaHCO₃ was added to a sugar mixture (approx. 500 μl) containing xylose and arabinose (xylose: approx. 10%, arabinose: approx. 1%). Thereupon, 30 μl arabinose dehydrogenase (activity of approx. 300 U/ml), 30 μl xylose reductase (activity of approx. 103 U/ml) and 2.5 μl NADH (concentration: 100 mM) were added. The mixture was incubated at 30° C. for approx. 20 minutes. 100% of the arabinose was reacted to arabonic acid. Thereby, 10% of the xylose contained in the mixture was converted into xylitol. The obtained solution was passed through a strong ion exchanger (Amberlyst A-26(OH), Alfa Aesar). Thereby, the resulting arabonic acid was separated completely from the sugar mixture.

### Example 5: Arabinose Oxidation with an Arabinose Dehydrogenase (Xylose Reductase for Cofactor Recycling), Xylose Reduction with a Xylose Reductase (Alcohol Dehydrogenase for Cofactor Recycling)

A sugar solution obtained from biomass (=xylan hydrolysate) was concentrated to a sugar concentration of approx. 63 g/l D-xylose and 7 g/l L-arabinose by evaporation, and pH=8.0 was adjusted with NaOH. 2.5 ml of 500 mM Tris-HCl buffer, pH=8.0, 200 U xylose reductase and 160 U arabinose dehydrogenase were added to 80 ml of said solution. In a 200 ml round-bottom flask, the solution was stirred with a magnetic stirrer (200 rpm) at 35° C. (water bath) for 20 minutes. The arabinose had been converted completely, and the solution now contained approx. 56 g/l D-xylose, approx. 7 g/l xylitol and approx. 7 g/l L-arabino-1,4-lactone/L-arabonic acid.

In said example, the employed cofactor was present in the employed enzyme lysates already to a sufficient extent and did not have to be added separately.

### Example 6: Conversion of a Mixture of Xylose and Arabinose (according to the Invention)

16.9 mg NaHCO₃ was added to a sugar mixture (approx. 500 μl) containing xylose and arabinose (xylose: approx. 10%, arabinose: approx. 1%). Thereupon, 30 μl arabinose dehydrogenase (activity of approx. 300 U/ml with arabinose; the enzyme also exhibits a certain activity as a xylose dehydrogenase), 30 μl xylose reductase (activity of approx. 103 U/ml) and 2.5 μl NADH (concentration: 100 mM) were added. The mixture was incubated at 30° C. for approx. 72 hours. 100% of the arabinose was reacted to arabonic acid, 45% of the xylose was reacted to xylonic acid, and 55% of the xylose was reacted to xylitol. The obtained solution was passed through a strong ion exchanger (Amberlyst A-26(OH), Alfa Aesar). Thereby, the arabonic acid and the xylonic acid which resulted were separated completely from the mixture.

This example demonstrates an at least partly parallel course of the first stage and the second stage of the process according to the invention: The first stage is the oxidation of the arabinose and the reduction of an equimolar part of the xylose. The second stage, which proceeds at least partly in parallel thereto, comprises the oxidation of half of the unreacted xylose by means of the activity of the arabinose dehydrogenase and the reduction of the other half of the remaining xylose.

### Example 7: Conversion of a Mixture of Xylose and Glucose (According to the Invention)

The reaction batch included the following components: 364 μl dH₂O, 2.5 μl NADPH-solution (100 mM), 10 μl D-glucose solution (50% w/v), 100 μl D-xylose solution (50% w/v), 5 μl glucose dehydrogenase (300 U/ml, measured with glucose), 19 μl xylose reductase (160 U/ml), and 5.6 mg CaCO₃. The glucose dehydrogenase which is used also exhibits a certain xylose dehydrogenase activity. The reaction was gently agitated at 35° C., and samples were taken at different points in time. By means of GC/MS, the content of sugars as well as of reaction products was determined. After 1 h, the glucose had, for the most part, been converted into gluconic acid. Likewise, a small part of the employed xylose (approx. 10%) had been oxidized to xylonic acid by this point in time. Stoichiometrically to the formed sugar acids, xylose had been reduced to xylitol after 1 h. Approximate composition of the reaction after 1 h: 10 mg/ml gluconic acid, 10 mg/ml xylonic acid, 20 mg/ml xylitol, 70 mg/ml xylose.

After 6 h, the xylose had been converted by approx. 90%. In comparison to the point in time after 1 h, the products xylonic acid and xylitol were thereby formed stoichiometrically. Approximate composition of the reaction after 6 h: 10 mg/ml gluconic acid, 40 mg/ml xylonic acid, 50 mg/ml xylitol, 10 mg/ml xylose.

### Example 8: Analysis of the Reactions by Means of GC/MS

For analyzing the oxidation reactions on the GC/MS, substrates and products were derivatized. For this purpose, 4 μl of the samples were transferred into a glass vial and dried in the Speedvac. For derivatization, 150 μl pyridine and 50 μl of a 99:1-mixture of N,O-bis(trimethylsilyl)trifluoroacetamide and trimethylchlorosilane were then added. Derivatization took place at 60° C. for 16 h. Subsequently, the samples were analyzed by GC-MS. In doing so, the samples were separated via the separation column HP-5 ms (5% phenyl)methylpolysiloxane in a gas chromatograph and analyzed in the mass spectrometer GCMS QP2010 Plus of Shimadzu.

### Non-Patent Literature

Berrocal A., Baezal J., Rodriguez J., Espinosa M., and Freer J. (2004) Effect of tree age on variation of Pinus radiate D. Don. chemical composition. J. Chil. Chem. Soc. 49(3), 251-256 (http://www.scielo.cl/scielo.php?script=sci_arttext&pid=S0717-97072004000300012, queried on Jun. 10, 2016)

Brodeur, G., Yau, E., Badal, K., Collier, J., Ramachandran, K., and Ramakrishnan, S. (2011). Chemical and Physicochemical Pretreatment of Lignocellulosic Biomass: A Review. Enzyme Research 2011.

Buchert J., Puls J., and Poutanen K. (1988). Comparison of Pseudomonas fragi and Gluconobacter oxydans for production of xylonic acid from hemicellulose hydrolysates. Applied and Environmental Microbiology 28, 367-372.

Johnsen, U., Dambeck, M., Zaiss, H., Fuhrer, T., Soppa, J., Sauer, U., and Schönheit, P. (2009). d-Xylose Degradation Pathway in the Halophilic Archaeon Haloferax volcanii. Journal of Biological Chemistry 284, 27290-27303.

Ko, B., Kim, J., and Kim, J. (2006). Production of Xylitol from D-Xylose by a Xylitol Dehydrogenase Gene-Disrupted Mutant of Candida tropicalis. Applied and Environmental Microbiology 72, 4207-4213.

Nidetzky, B., Neuhauser, W., Haltrich, D., and Kulbe, K. (1996). Continuous enzymatic production of xylitol with simultaneous coenzyme regeneration in a charged membrane reactor. Biotechnology and Bioengineering 52, 387-396.

Stephens, C., Christen, B., Fuchs, T., Sundaram, V., Watanabe, K., and Jenal, U. (2006). Genetic Analysis of a Novel Pathway for D-Xylose Metabolism in Caulobacter crescentus. Journal of Bacteriology 189, 2181-2185.

Toivari, M., Nygård, Y., Penttilä, M., Ruohonen, L., and Wiebe, M. (2012a). Microbial d-xylonate production. Applied Microbiology and Biotechnology 96, 1-8.

Toivari, M., Nygård, Y., Kumpula, E. -P., Vehkomäki, M. -L., Benčina, M., Valkonen, M., Maaheimo, H., Andberg, M., Koivula, A., Ruohonen, L., et al. (2012b). Metabolic engineering of Saccharomyces cerevisiae for bioconversion of d-xylose to d-xylonate. Metabolic Engineering 14.

Watanabe, S., Shimada, N., Tajima, K., Kodaki, T., and Makino, K. (2006). Identification and Characterization of 1-Arabonate Dehydratase, 1-2-Keto-3-deoxyarabonate Dehydratase, and 1-Arabinolactonase Involved in an Alternative Pathway of 1-Arabinose Metabolism NOVEL EVOLUTIONARY INSIGHT INTO SUGAR METABOLISM. Journal of Biological Chemistry 281, 33521-33536.

Zhang, Y., Gao, F., Zhang, S. -P., Su, Z. -G., Ma, G. -H., and Wang, P. (2011). Simultaneous production of 1,3-dihydroxyacetone and xylitol from glycerol and xylose using a nanoparticle-supported multi-enzyme system with in situ cofactor regeneration. Bioresource Technology 102, 1837-1843.

1. A process for obtaining n+a oxidation and reduction products from a mixture of n sugars selected from the group consisting of C5 and C6 sugars,
wherein n is at least 2 and a is at least 1,
wherein at least two of the sugars in the mixture are present at a non-equimolar ratio to each other,
wherein, in a first stage, at least one of the sugars which are present at a non-equimolar ratio to each other is oxidized enzymatically and, at the same time, at least one of the other sugars present at a non-equimolar ratio to each other is reduced enzymatically, and
wherein, in the first stage, a portion of at least one of the sugars present at a non-equimolar ratio to each other is not converted,
characterized in that, in at least a second stage, at least a portion of the sugar not converted in the first stage is oxidized enzymatically by half and, respectively, is reduced enzymatically by the remaining half.

2. A process according to claim 1, characterized in that sugar acids and sugar acid lactones, respectively, are obtained as oxidation products, and sugar alcohols are obtained as reduction products.

3. A process according to claim 1, characterized in that the mixture of sugars contains xylose and arabinose, with xylose being present in excess.

4. A process according to claim 3, characterized in that arabinose is oxidized to arabonic acid or to arabonic acid lactone, respectively, and a portion of the xylose is reduced to xylitol in the first stage, and, in the second stage, the unreacted xylose is oxidized completely or partly to xylonic acid or to xylonolactone by half and, respectively, the remaining half is reduced to xylitol.

5. A process according to claim 3, characterized in that arabonic acid which has formed and/or xylonic acid which has formed is/are processed further into α-ketoglutaric acid.

6. A process according to claim 3, characterized in that the mixture additionally contains glucose.

7. A process according to claim 1, characterized in that the mixture contains glucose in excess relative to the other existing sugar(s), and sorbitol is obtained at least partly from the glucose.

8. A process according to claim 1, characterized in that, at least in one of the two stages, preferably at least in the second stage, particularly preferably both in the first and in the second stage, at least one redox cofactor and at least one enzyme dependent on said redox cofactor are present in the reaction mixture.

9. A process according to claim 8, characterized in that the redox cofactor(s) is/are regenerated by enzymatic reactions proceeding in parallel.

10. A process according to claim 1, characterized in that the first and the second stage are performed in a one-pot reaction.

11. A process according to claim 10, characterized in that the two stages proceed at least partly simultaneously.

12. A process according to claim 1, comprising the removal of accumulating sugar acids from the mixture.

13. A process according to claim 1, characterized in that the mixture containing the sugars has been obtained from a hemicellulose-containing material.

14. A process according to claim 13, characterized in that the hemicellulose-containing material has been obtained by pulping a lignocellulosic material.

15. A process according to claim 14, characterized in that the lignocellulosic material is a material selected from straw and/or husks.

16. A process according to claim 14, characterized in that the lignocellulosic material has been obtained by pulping with an alcohol.

17. A process according to claim 9, characterized in that the redox cofactor(s) is/are regenerated by enzymatic reactions both in the first and in the second stage.

18. A process according to claim 15, characterized in that the lignocellulosic material is selected from the group consisting of wheat straw, bagasse, energy grasses, elephant grass, switch grass, and lemmas.

19. A process according to claim 16, characterized in that the lignocellulosic material has been obtained by pulping with a C₁₋₄ alcohol, water and an alkali.
